(19) United States
(12) Patent Application Publication
(10) Pub. No.: US 2026/0325478 A1
(43) Pub. Date: Oct. 22, 2026

(54) ENDURANCE ENHANCER

(71) Applicant: Nissui Corporation, Minato-ku, Tokyo (JP)

(72) Inventors: Eisuke OCHI, Tokyo (JP); Kaori YOKOI, Tokyo (JP); Kenichi YANAGIMOTO, Tokyo (JP)

(73) Assignee: Nissui Corporation, Minato-ku, Tokyo (JP)

(21) Appl. No.: 18/881,003

(22) PCT Filed: Jul. 4, 2023
(86) PCT No.: PCT/JP2023/024722

§ 371 Date: Jan. 3, 2025

(30) Foreign Application Priority Data
Jul. 4, 2022 (JP) 2022-107740

(51) Int. Cl. A61K 31/232; A61K 35/60; A61P 43/00

(52) U.S. Cl. CPC A61K 31/232; A61K 35/60; A61P 43/00

(57) ABSTRACT

The present invention provides: an endurance-enhancing composition including, as an active ingredient, a triglyceride containing a highly unsaturated fatty acid and a medium-chain fatty acid as constituent fatty acids; an endurance-enhancing food composition or food additive including the former composition; and others.

### TECHNICAL FIELD

The present invention relates to an endurance-enhancing composition and a method of enhancing endurance by using the composition.

### BACKGROUND ART

Highly unsaturated fatty acids such as eicosapentaenoic acid (EPA) and docosahexaenoic acid (DHA) contained in fish oil are known to have various physiological effects. For example, EPA is known to exert an effect of ameliorating dyslipidemia (hyperlipidemia) such as hypertriglyceridemia. Thus, these highly unsaturated fatty acids are widely used as, for instance, pharmaceuticals, health foods, and food materials.

When utilized in pharmaceuticals, ethyl esters of highly unsaturated fatty acids are used, and when utilized in health foods, for example, triglycerides such as refined fish oil are often used.

Based on the knowledge that medium-chain fatty acids are fatty acids that are better absorbed, there are reports on efforts to increase the absorption of highly unsaturated fatty acids by using triglycerides containing both highly unsaturated fatty acids and medium-chain fatty acids.

PTL 1 describes a composition that is suitable for parenteral administration and can be used for treatment or prevention of atherosclerosis and hyperlipidemia, the composition comprising triglycerides containing 1-20% of C₂₀₋₂₂ fatty acids, 30-50% of C₁₄₋₁₈ fatty acids, and 40-60% of C₆₋₁₂ fatty acids, as constituent fatty acids.

PTL 2 describes that triglycerides made by transesterification of fish oil and medium-chain fatty acid triglycerides are suitable as a nutritional substitute for patients with reduced resistance to infection and others because of their fast absorption.

PTL 3 describes a fatty oil composition with a blood lipid regulating function, which composition comprises triglycerides containing 5-55% of n-3 long-chain polyunsaturated fatty acids and 2-40% of medium-chain fatty acids.

PTLs 4-6 describe that in view of the finding that a fatty acid linked to position 1 or 3 of triglyceride is less easily degraded by lipase and less easily absorbed, triglycerides with a highly unsaturated fatty acid linked at position 2 and medium-chain fatty acids linked at positions 1 and 3 are suitable for infusions and enteral nutrition preparations for patients with reduced absorption.

PTL 7 describes that if triglycerides (EPA/MCT structured lipids) containing fatty acids derived from fish oil including EPA and medium-chain fatty acids as constituent fatty acids are ingested, EPA is absorbed faster and a blood EPA concentration is maintained at a higher level than ingestion of mixed oil consisting of refined fish oil and triglycerides containing medium-chain fatty acids as constituent fatty acids. However, PTL 7 merely shows that a single intake of EPA/MCT structured lipids transiently increases the transfer of EPA into the blood, but does not show what kinds of effects can be obtained by continuous administration.

PTL 8 discloses that oral intake of highly unsaturated fatty acids increases lymphatic fluid flow, and that intake of triglycerides containing both a highly unsaturated fatty acid and a medium-chain fatty acid in a single molecule as constituent fatty acids increases lymphatic fluid flow more significantly.

Several studies have been reported for the effects of ingesting fish oil or n-3 (or omega-3) long-chain unsaturated fatty acids (PUFA) such as EPA or DHA on exercise performance. While some reports concluded that n-3 PUFA intake had a beneficial effect on exercise performance, others reported that exercise performance was not improved.

NPL1 describes that healthy male cyclists were fed with 3.2 g/day of n-3 PUFA (0.8 g EPA and 2.4 g DHA) for 8 weeks and exercised on a bicycle ergometer for at least 60 minutes, and that, although heart rate, RPP (Rate Pressure Product), and whole-body O₂ consumption decreased, there was no significant difference in exercise time until fatigue when compared to placebo.

NPL 2 describes that when male cyclists were fed with 6 g/day of fish oil with or without vitamin E for 3 weeks and exercised on a bicycle ergometer for about 1 hour, there was no significant difference in exercise performance when compared to placebo.

NPL 3 reports that 14 days of fish oil intake (containing 1.1 g/day of EPA and 0.7 g/day of DHA) by healthy, sedentary male subjects had no effect on maximum oxygen uptake (VO₂ₘₐₓ) and energy metabolism in a human study using a bicycle ergometer.

NPL 4 describes that when elite cyclists were fed with 2.6 g/day of n-3 PUFA for 3 weeks and a bicycle pedaling test was conducted, there was a significant increase in maximum oxygen uptake (VO₂ₘₐₓ) compared to that of placebo.

NPL 5 describes that maximum oxygen uptake was significantly improved in overweight women after 8 weeks of n-3 PUFA intake and lifestyle modification when compared to the control group.

NPL 6 describes that subjects were fed with 3.6 g/day of fish oil (29.6% EPA and 12.9% DHA based on total fatty acids) for 8 weeks without strenuous exercise and then exercised using a bicycle ergometer, and that the oxygen uptake was lower and the numerical value for the Borg Scale, a measure of subjective exercise intensity, was also lower than those of placebo.

However, NPLs 4-6 neither disclose specific results showing that intake of fish oil or n-3 PUFAs improved exercise performance, nor do they describe any results showing endurance enhancement such as prolonged exercise duration.

### CITATION LIST

### Patent Literature

PTL 1: Japanese Patent Laid-Open No. S63-154618
PTL 2: Japanese Patent No. 2722229
PTL 3: Japanese Patent Laid-Open No. 2001-226693
PTL 4: U.S. Pat. No. 4,607,052
PTL 5: WO88/09325
PTL 6: WO90/04010
PTL 7: WO2015/050071
PTL 8: WO2019/156210

### Non Patent Literature

NPL 1: Peoples G E, et al., Journal of Cardiovascular Pharmacology, 52 (6), p. 540-547 (2008)
NPL 2: Oostenbrug G S, et al., Journal of Applied Physiology, 83 (3), p. 746-752 (1997)
NPL 3: Bortolotti M, et al., Clinical Nutrition, 26 (2), p. 225-230 (2007)
NPL 4: Zebrowska A, et al., European Journal of Sport Science, 15 (4), p. 305-314 (2015)
NPL 5: Haghravan S, et al., Archives of Iranian Medicine, 19 (5), p. 342-347 (2016)
NPL 6: Kawabata F, et al., Bioscience, Biotechnology, and Biochemistry, 78 (12), p. 2081-2088 (2014)

### SUMMARY OF INVENTION

### Technical Problem

An object of the present invention is to provide a means for enhancing endurance.

### Solution to Problem

In the course of developing new applications of triglycerides (structured lipids), which contain a highly unsaturated fatty acid and a medium-chain fatty acid as constituent fatty acids in a single molecule, the present inventors have unexpectedly found that the intake of the structured lipids significantly prolonged the duration of exercise until fatigue during maximum oxygen uptake (VO₂peak) measurement when compared with that before intake. The present inventors have also found that the change in exercise duration between before and after intake of the test substance was significantly greater in the group that ingested the structured lipids than in the group that ingested a mixture (physically mixed oil) of triglycerides that have similar fatty acid components and do not contain a highly unsaturated fatty acid and a medium-chain fatty acid in a single molecule. Further, the anaerobic exercise threshold (AT), i.e., the transition point from aerobic to anaerobic exercise, was also found to be significantly higher after intake of the structured lipids than that before the intake or after intake of the physically mixed oil. Furthermore, it has been found that in the group of subjects who ingested the structured lipids, the change in exercise duration between before and after intake of the test substance tended to be greater in subjects with low fishery product intake than in subjects with high fishery product intake.

Based on these findings, the present inventors have conducted further intensive research and completed the present invention.

Specifically, the present invention is as follows.
[1] An endurance-enhancing composition comprising, as an active ingredient, a triglyceride containing a highly unsaturated fatty acid and a medium-chain fatty acid as constituent fatty acids.
[2] The composition according to [1], wherein the highly unsaturated fatty acid accounts for 10 to 70 wt % in fatty acid makeup of the composition and the medium-chain fatty acid accounts for 20 to 70 wt % in fatty acid makeup of the composition.
[3] The composition according to [1] or [2], wherein the composition is used such that 19 mg/kg body weight/day or more and 30 mg/kg body weight/day or less of the highly unsaturated fatty acid is ingested by a subject for 8 weeks or longer.
[4] The composition according to any one of [1] to [3], wherein the composition is used so as to be ingested by a subject whose fishery product intake is 1.1 kcal/kg body weight/day or less.
[5] The composition according to any one of [1] to [4], wherein the composition is used so as to be ingested by a subject whose maximum oxygen uptake is 60 mL/kg/min or less.
[6] The composition according to any one of [1] to [5], wherein the highly unsaturated fatty acid is a fatty acid with 20 or more carbon atoms.
[7] The composition according to [6], wherein the highly unsaturated fatty acid is eicosapentaenoic acid or docosahexaenoic acid.
[8] The composition according to any one of [1] to [7], wherein the medium-chain fatty acid is a C₈₋₁₂ fatty acid.
[9] The composition according to any one of [1] to [8], wherein enhancement of the endurance is at least one selected from the group consisting of prolonged exercise duration, prolonged exercise duration during high-intensity exercise, prolonged exercise duration during aerobic exercise, prolonged exercise duration during incremental load exercise, accelerated lipid burning during exercise, accelerated lipid burning during high-intensity exercise, accelerated lipid burning during aerobic exercise, accelerated lipid burning during incremental load exercise, increased anaerobic exercise threshold, reduced fatigue during exercise, and enhanced muscle endurance.
[10] An endurance-enhancing food composition or food additive comprising the composition according to any one of [1] to [9].
[11] A method for producing a food composition or food additive used to enhance endurance, comprising imparting an endurance-enhancing function to the food composition or the food additive by blending the composition according to any one of [1] to [9] into the food composition or the food additive.
[12] A method of enhancing endurance, comprising feeding a subject with an effective amount of a composition including, as an active ingredient, a triglyceride containing a highly unsaturated fatty acid and a medium-chain fatty acid as constituent fatty acids.
[13] The method according to [12], wherein the highly unsaturated fatty acid accounts for 10 to 70 wt % in fatty acid makeup of the composition and the medium-chain fatty acid accounts for 20 to 70 wt % in fatty acid makeup of the composition.
[14] The method according to [12] or [13], wherein 19 mg/kg body weight/day or more and 30 mg/kg body weight/day or less of the highly unsaturated fatty acid is ingested by a subject for 8 weeks or longer.
[15] The method according to any one of [12] to [14], wherein the subject has a fishery product intake of 1.1 kcal/kg body weight/day or less.
[16] The method according to any one of [12] to [15], wherein the subject has a maximum oxygen uptake of 60 mL/kg/min or less.
[17] The method according to any one of [12] to [16], wherein the highly unsaturated fatty acid is a fatty acid with 20 or more carbon atoms.
[18] The method according to [17], wherein the highly unsaturated fatty acid is eicosapentaenoic acid or docosahexaenoic acid.
[19] The method according to any one of [12] to [18], wherein the medium-chain fatty acid is a C₈₋₁₂ fatty acid.
[20] The method according to any one of [12] to [19], wherein enhancement of the endurance is at least one selected from the group consisting of prolonged exercise duration, prolonged exercise duration during high-intensity exercise, prolonged exercise duration during aerobic exercise, prolonged exercise duration during incremental load exercise, accelerated lipid burning during exercise, accelerated lipid burning during high-intensity exercise, accelerated lipid burning during aerobic exercise, accelerated lipid burning during incremental load exercise, increased anaerobic exercise threshold, reduced fatigue during exercise, and enhanced muscle endurance.
[21] The method according to any one of [12] to [20], wherein the composition is provided in a form of a food composition or food additive.
[22] A composition for use in enhancing endurance, which composition comprising, as an active ingredient, a triglyceride containing a highly unsaturated fatty acid and a medium-chain fatty acid as constituent fatty acids.
[23] The composition according to [22], wherein the highly unsaturated fatty acid accounts for 10 to 70 wt % in fatty acid makeup of the composition and the medium-chain fatty acid accounts for 20 to 70 wt % in fatty acid makeup of the composition.
[24] The composition according to [22] or [23], wherein the composition is used such that 19 mg/kg body weight/day or more and 30 mg/kg body weight/day or less of the highly unsaturated fatty acid is ingested by a subject for 8 weeks or longer.
[25] The composition according to any one of [22] to [24], wherein the composition is used so as to be ingested by a subject whose fishery product intake is 1.1 kcal/kg body weight/day or less.
[26] The composition according to any one of [22] to [25], wherein the composition is used so as to be ingested by a subject whose maximum oxygen uptake is 60 mL/kg/min or less.
[27] The composition according to any one of [22] to [26], wherein the highly unsaturated fatty acid is a fatty acid with 20 or more carbon atoms.
[28] The composition according to [27], wherein the highly unsaturated fatty acid is eicosapentaenoic acid or docosahexaenoic acid.
[29] The composition according to any one of [22] to [28], wherein the medium-chain fatty acid is a C₈₋₁₂ fatty acid.
[30] The composition according to any one of [22] to [29], wherein enhancement of the endurance is at least one selected from the group consisting of prolonged exercise duration, prolonged exercise duration during high-intensity exercise, prolonged exercise duration during aerobic exercise, prolonged exercise duration during incremental load exercise, accelerated lipid burning during exercise, accelerated lipid burning during high-intensity exercise, accelerated lipid burning during aerobic exercise, accelerated lipid burning during incremental load exercise, increased anaerobic exercise threshold, reduced fatigue during exercise, and enhanced muscle endurance.
[31] The composition according to any one of [22] to [30], which is provided in a form of a food composition or food additive.
[32] Use of a composition comprising, as an active ingredient, a triglyceride containing a highly unsaturated fatty acid and a medium-chain fatty acid as constituent fatty acids for the manufacture of an endurance-enhancing composition.
[33] The use according to [32], wherein the highly unsaturated fatty acid accounts for 10 to 70 wt % in fatty acid makeup of the composition and the medium-chain fatty acid accounts for 20 to 70 wt % in fatty acid makeup of the composition.
[34] The use according to [32] or [33], wherein the endurance-enhancing composition is used such that 19 mg/kg body weight/day or more and 30 mg/kg body weight/day or less of the highly unsaturated fatty acid is ingested by a subject for 8 weeks or longer.
[35] The use according to any one of [32] to [34], wherein the endurance-enhancing composition is used so as to be ingested by a subject whose fishery product intake is 1.1 kcal/kg body weight/day or less.
[36] The use according to any one of [32] to [35], wherein the endurance-enhancing composition is used so as to be ingested by a subject whose maximum oxygen uptake is 60 mL/kg/min or less.
[37] The use according to any one of [32] to [36], wherein the highly unsaturated fatty acid is a fatty acid with 20 or more carbon atoms.
[38] The use according to [37], wherein the highly unsaturated fatty acid is eicosapentaenoic acid or docosahexaenoic acid.
[39] The use according to any one of [32] to [38], wherein the medium-chain fatty acid is a C₈₋₁₂ fatty acid.
[40] The use according to any one of [32] to [39], wherein enhancement of the endurance is at least one selected from the group consisting of prolonged exercise duration, prolonged exercise duration during high-intensity exercise, prolonged exercise duration during aerobic exercise, prolonged exercise duration during incremental load exercise, accelerated lipid burning during exercise, accelerated lipid burning during high-intensity exercise, accelerated lipid burning during aerobic exercise, accelerated lipid burning during incremental load exercise, increased anaerobic exercise threshold, reduced fatigue during exercise, and enhanced muscle endurance.
[41] The use according to any one of [32] to [40], wherein the endurance-enhancing composition is provided in a form of a food composition or food additive.

### Advantageous Effect of Invention

Oral intake of the composition of the present invention can enhance endurance.

### BRIEF DESCRIPTION OF DRAWINGS

FIG. 1 shows an exercise duration during maximum oxygen uptake measurement before and after intake in the group of ingesting triglycerides (STG) containing fish oil-derived fatty acids including EPA and medium-chain fatty acids, as constituent fatty acids (STG group), or in the group of ingesting mixed oil (PM) prepared by mixing refined fish oil and MCT at a weight ratio of 50:50 (PM group). *: p<0.05 for the difference between pre and post intake values in the STG group.

FIG. 2 shows a change in exercise duration during maximum oxygen uptake measurement between before and after intake in the STG or PM group. *: p<0.05 for the difference in the change between the STG group and the PM group.

FIG. 3 shows values for anaerobic exercise threshold (% VO₂peak) before and after intake in the STG or PM group. **: p<0.01 for the difference between pre and post intake values in the STG group. †: p<0.05 for the difference between the post intake value of the STG group and the post intake value of the PM group.

FIG. 4 shows a change in the value for anaerobic exercise threshold (% VO₂peak) between before and after intake in the STG or PM group. *: p<0.05 for the difference in the change between the STG group and the PM group.

### DESCRIPTION OF EMBODIMENTS

Hereinafter, the present invention will be described more specifically.

Note that the following abbreviations may be herein used.
EPA: eicosapentaenoic acid
DHA: docosahexaenoic acid
MCFA: medium-chain fatty acid
MCT: triglyceride exclusively containing a medium-chain fatty acid(s) as a constituent fatty acid(s)
STG: structured triglyceride, structured triacylglycerol, or structured lipid (in the Examples, a structured lipid with specific fatty acid makeup is referred to as STG)
PM: physical mixture or physically mixed oil (in the Examples, physically mixed oil with specific fatty acid makeup is referred to as PM)
AT: anaerobic exercise threshold (also called anaerobic metabolic threshold)

The present invention provides an endurance-enhancing composition comprising, as an active ingredient, a triglyceride containing a highly unsaturated fatty acid and a medium-chain fatty acid as constituent fatty acids (hereinafter, sometimes referred to as a composition of the present invention).

As used herein, the term “endurance” refers to the ability to continuously perform a specific physical activity such as exercise (e.g., walking, running, swimming, pedaling a bicycle). The term “endurance enhancement” means that the duration of the physical activity is prolonged, or that the body's energy metabolism is improved so that the duration of the physical activity can be prolonged.

The endurance enhancement in the present invention can be evaluated by the multistep load incremental method (Whipp B J et al., J. Apple. Physiol.: Respirat. Environ. Exercise physiol. 50 (1): 217-221, 1981). For example, a bicycle ergometer may be used. In this case, the pedal rotation speed during exercise is set to 60 rpm. After 3 minutes of rest, 3 minutes of warm-up exercise at 20-watt load is performed. Then, exercise is started with 60-watt load. The load is increased by 30 watt every 2 minutes. The exercise is made to continue until the point of fatigue/exhaustion. The final exercise load is defined at the time when the subject's pedal rotation speed falls below 50 rpm three times. At the time point when oxygen uptake measurement at that load is completed, the exercise and the measurement should be ended. If the exercise duration is significantly prolonged in the group of ingesting a test substance when compared to the group before or without ingestion of the test substance, the test substance is considered to exert an endurance-enhancing effect.

In addition, if the anaerobic exercise threshold (AT; also referred to as an anaerobic metabolic threshold) is significantly higher in the group of ingesting a test substance than in the group before or without ingestion of the test substance, the test substance is also considered to exert an endurance-enhancing effect. As used herein, AT is the exercise intensity at which the transition from aerobic exercise to anaerobic exercise occurs in the multistep load incremental method (incremental load test) as described above, and is identified by measuring the carbon dioxide output. Thus, as used herein, AT may be paraphrased as the ventilatory exercise threshold (VT). As used herein, AT is expressed by the percentage ratio (% VO₂peak) of the oxygen uptake to the maximum oxygen uptake at the exercise intensity at which aerobic exercise switches to anaerobic exercise. The % VO₂peak can be calculated by measuring oxygen uptake and identifying AT by measuring carbon dioxide output in the multistep load incremental method described above. Throughout this specification, the value of % VO₂peak, which represents the anaerobic exercise threshold, is sometimes referred to as the “AT level”.

In one embodiment, the endurance enhancement in the present invention may be prolonged exercise duration. Exercise duration refers to the time from the start of exercise to the point of fatigue/exhaustion and difficulty in continuing the exercise in a specific physical activity. The prolonged exercise duration can be, for example, prolonged exercise duration during high-intensity exercise, prolonged exercise duration during aerobic exercise, or prolonged exercise duration during incremental load exercise. The high-intensity exercise is defined as exercise at an intensity of 70% VO₂max or more, e.g., 75% VO₂max or more, 80% VO₂max or more, 85% VO₂max or more, 90% VO₂max or more, 95% VO₂max or more, or 100% VO₂max. The VO₂max is the maximum oxygen uptake and the maximum amount of oxygen that can be taken in per minute (mL/kg/min or L/min). The % VO₂max is the percentage ratio of the subject's oxygen uptake to the VO₂max during exercise. The aerobic exercise is exercise that uses carbohydrates and lipids as energy sources through aerobic metabolism. The incremental load exercise refers to exercise performed under conditions in which the exercise load is increased by a certain amount at regular intervals.

Note that the prolonged exercise duration may be influenced by various factors such as heart rate during exercise, muscle damage, and improvement of muscle fatigue. Therefore, results such as an increase in maximum oxygen uptake (VO₂max), for example, do not indicate actual prolonged exercise duration.

In one embodiment, the endurance enhancement in the present invention may be the improvement of energy metabolism in the body so that the duration of exercise can be prolonged. Such an improvement in the body's energy metabolism can be accelerated lipid burning during exercise. The “lipid burning” refers to the use of lipids as an energy source. The accelerated lipid burning during exercise may be, for example, accelerated lipid burning during high-intensity exercise, accelerated lipid burning during aerobic exercise, or accelerated lipid burning during incremental load exercise. The effect of the composition of the present invention to enhance AT means that lipid burning during exercise is accelerated. Conventionally, medium-chain fatty acids are known to be used more efficiently as an energy source during exercise than long-chain fatty acids such as EPA (Am J Clin Nutr. 36 (5), 950-62. (1982)). On the other hand, it is not known that lipid burning during exercise is accelerated by ingestion of long-chain fatty acids alone, which are not in the form of structured lipids. In fact, as shown in the Examples below, AT was not increased using the physically mixed oil comprising triglycerides containing EPA as a constituent fatty acid. When a triglyceride containing a highly unsaturated fatty acid and a medium-chain fatty acid as constituent fatty acids is ingested, the body's energy metabolism will be improved, thereby accelerating lipid burning during exercise. This has not been previously known in the art.

In one embodiment, the endurance enhancement in the present invention can be an increase in AT. The increase in AT may be, for example, an increase in AT during high-intensity exercise, an increase in AT during aerobic exercise, or an increase in AT during incremental load exercise.

In one embodiment, the endurance enhancement in the present invention can be reduced fatigue during exercise. As used herein, the term “fatigue” means physical fatigue caused by exercise. The prolonged exercise duration due to the ingestion of the composition of the present invention may be attributed to the fact that fatigue less easily occurs. The reduced fatigue during exercise may be, for example, reduced fatigue during high-intensity exercise, reduced fatigue during aerobic exercise, or reduced fatigue during incremental load exercise.

In one embodiment, the endurance enhancement in the present invention can be enhanced muscle endurance. The muscle endurance is the ability of a particular muscle to contract repeatedly, or the ability to sustain an exercise using some muscles of the body for a long time. The bicycle ergometer-use exercise used in the Examples is known as training aiming at enhancing muscle endurance. The enhanced muscle endurance may be, for example, enhanced muscle endurance during high-intensity exercise, enhanced muscle endurance during aerobic exercise, or enhanced muscle endurance during incremental load exercise.

In the present invention, the highly unsaturated fatty acid is a fatty acid with 18 or more carbon atoms and 3 or more double bonds, for example, a fatty acid with 20 or more carbon atoms and 3 or more double bonds, a fatty acid with 20 or more carbon atoms and 4 or more double bonds, or a fatty acid with 20 or more carbon atoms and 5 or more double bonds. Specific examples include α-linolenic acid (18:3,n-3), γ-linolenic acid (18:3,n-6), octadecatetraenoic acid (18:4,n-3), icosatetraenoic acid (20:4,n-3), arachidonic acid (20:4,n-6), dihomo-γ-linolenic acid (20:3,n-6), icosapentaenoic acid (20:5,n-3), henicosapentaenoic acid (21:5,n-3), docosapentaenoic acid (22:5,n-6), or docosahexaenoic acid (22:6,n-3). In a preferred embodiment, the highly unsaturated fatty acid is n-3 highly unsaturated fatty acid, more preferably eicosapentaenoic acid and/or docosahexaenoic acid.

Glycerides containing a highly unsaturated fatty acid as a constituent fatty acid are known to be abundant in certain microbial oils, plant oils, marine animal oils, and the like. Specific examples include: fish oils such as sardine oil, tuna oil, bonito oil, menhaden oil, cod liver oil, herring oil, capelin oil, and salmon oil; marine animal oils from crustaceans such as krill; plant oils such as perilla, flaxseed, soybean, and rapeseed oil; or fatty oils produced by microorganisms belonging to the genus Mortierella, Penicillium, Aspergillus, Rhodotorula, or Fusarium; and the like. In one embodiment, the highly unsaturated fatty acid, namely a constituent fatty acid of the triglyceride in the present invention may be derived from these fatty oils.

In the present invention, the medium-chain fatty acid refers to that with 8 to 12 carbon atoms, and may be either a saturated or unsaturated fatty acid. Specific examples include caprylic acid, pelargonic acid, capric acid, lauric acid, and the like. One kind of the medium-chain fatty acid may be used, or two or more kinds thereof may be used in combination. In a preferred embodiment, the medium-chain fatty acid is caprylic acid and/or capric acid. Glycerides containing a medium-chain fatty acid as a constituent fatty acid are known to be abundant in coconut oil, palm oil, and the like. In one embodiment, the medium-chain fatty acid, namely a constituent fatty acid of the triglyceride in the present invention may be derived from these fatty oils.

The active ingredient in the composition of the present invention is a triglyceride containing a highly unsaturated fatty acid and a medium-chain fatty acid as constituent fatty acids. As shown in the Examples below, a triglyceride containing both a highly unsaturated fatty acid and a medium-chain fatty acid as constituent fatty acids in a single molecule elicits a higher endurance-enhancing effect than mixed oils with an equivalent fatty acid makeup.

Such a triglyceride may be prepared, for example, by subjecting a triglyceride containing a highly unsaturated fatty acid as a constituent fatty acid and a triglyceride containing a medium-chain fatty acid as a constituent fatty acid to a transesterification reaction, as described above. The catalyst used for transesterification may be either a chemical catalyst or an enzyme (lipase). In the transesterification using a chemical catalyst, the constituent fatty acids are linked at random positions. Note that such a triglyceride prepared by changing the positions of linkage of fatty acids and by changing fatty acid makeup in a naturally occurring triglyceride is sometimes called a structured lipid.

In the case of using a chemical catalyst, for example, about 0.1 to 2 wt % of the catalyst is added to an oil obtained by mixing a triglyceride containing a highly unsaturated fatty acid as a constituent fatty acid and a triglyceride containing a medium-chain fatty acid as a constituent fatty acid. The reaction is then carried out under normal or reduced pressure at 50 to 270° C. for 3 to 120 minutes with stirring. The target product can be obtained by routine purification procedures such as water washing, drying, decolorization, and deodorization. The blended ratio between the triglyceride containing a highly unsaturated fatty acid as a constituent fatty acid and the triglyceride containing a medium-chain fatty acid as a constituent fatty acid may be set as appropriate so as to exert an endurance-enhancing effect. For example, the mixing is performed so that the ratio between the highly unsaturated fatty acid and the medium-chain fatty acid in the fatty acid makeup of the composition of the present invention is set to the ratio disclosed herein.

Examples of the chemical catalyst include: an alkali metal hydroxide such as lithium hydroxide, sodium hydroxide, or potassium hydroxide; an alkali metal alkoxide such as lithium methoxide or sodium methoxide; and the like.

In one embodiment, in the fatty acid makeup of the composition of the present invention, the highly unsaturated fatty acid may account for 10 to 70 wt %, for example, 15 to 50 wt % or 20 to 40 wt %, and the medium-chain fatty acid may account for 20 to 70 wt %, for example, 30 to 60 wt % or 40 to 55 wt %. In a preferred embodiment, in the fatty acid makeup of the composition of the present invention, the highly unsaturated fatty acid may account for 10 to 70 wt % and the medium-chain fatty acid may account for 20 to 70 wt %. Further, in one embodiment where the highly unsaturated fatty acid is EPA and DHA and the medium-chain fatty acid is caprylic acid and capric acid, the highly unsaturated fatty acid may account for 5 to 65 wt %, for example, 10 to 45 wt % or 15 to 35 wt %, and the medium-chain fatty acid may account for 20 to 70 wt %, for example, 30 to 60 wt % or 40 to 55 wt % in the fatty acid makeup of the composition of the present invention. In a preferred embodiment, the highly unsaturated fatty acid may be EPA and DHA, the medium-chain fatty acid may be caprylic acid and capric acid, and the highly unsaturated fatty acid may account for 5 to 65 wt % and the medium-chain fatty acid may account for 20 to 70 wt % in the fatty acid makeup of the composition of the present invention.

As used herein, the percentage (wt %) of each fatty acid with respect to the total fatty acids in the composition is calculated based on the values measured by gas chromatography after esterification of the components in the composition in accordance with “AOCS official method Celb-89”, unless otherwise specified. The fatty acid content also means the percentage (wt %) of such a fatty acid with respect to the total fatty acids mentioned above. The analytical conditions for gas chromatography are as follows.
Instrument: Agilent 6890N GC system (Agilent)
Column: DB-WAX (Agilent Technologies, 30 m×0.25 mm ID, 0.25 μm film thickness)
Carrier gas: Helium (1.0 mL/min, constant flow)
Inlet temperature: 250° C.
Injection volume: 1 μL
Injection method: Split
Split ratio: 20:1
Column Oven: 180° C.-3° C./min-230° C.
Detector: FID
Detector temperature: 250° C.

The subject to be administered with the composition of the present invention is a mammal, preferably a human. In one embodiment, the composition of the present invention may be administered to a subject in need of endurance enhancement, particularly prolonged exercise duration. When the composition of the present invention is used for non-therapeutic purposes, the subject of administration is a healthy subject, which includes a professional athlete or an athlete who trains him/herself routinely, a healthy individual or a casual athlete who is active in exercise or sports, or a healthy individual who does not routinely engage in high-intensity exercise.

In one embodiment, the subject of administration may be a person whose “high-intensity physical activity days at work per week”, which is a question item of the Global Physical Activity Questionnaire (GPAQ; Bull F C et al., Global physical activity questionnaire (GPAQ): nine-country reliability and validity study. J Phys Act Health. 2009 November; 6 (6): 790-804), are from 0 to 7 days, preferably from 0 to 2 days, for example, 0, 1, or 2 days.

In one embodiment, the subject of administration may be a person whose “moderate-intensity physical activity days at work per week”, which is a question item of the GPAQ, are from 0 to 7 days, preferably from 0 to 3 days, for example, 0, 1, 2, or 3 days.

Here, the “work” includes schoolwork and part-time jobs. In addition, the terms “high-intensity physical activity” and “moderate-intensity physical activity” refer to physical activity of 8 and 4 METs, respectively (Global Physical Activity Questionnaire (GPAQ) Analysis Guide, Surveillance and Population-Based Prevention, Prevention of Noncommunicable Diseases Department, World Health Organization).

In one embodiment, the subject of administration may be a person whose “days with travel of 10 minutes or longer per week”, which is a question item of the GPAQ, are from 0 to 7 days, preferably from 4 to 7 days, for example, 4, 5, 6, or 7 days.

In one embodiment, the subject of administration may be a person whose maximum oxygen uptake is 60 mL/kg/min or less, for example, from 40 to 60 mL/kg/min, from 40 to 50 mL/kg/min, or from 50 to 60 mL/kg/min.

As demonstrated in the Examples below, the composition of the present invention is highly effective in enhancing endurance in the subject of administration with low intake of fishery products. In the present invention, the fishery product(s) refers to fish, shellfish, crustaceans, and cephalopods, but does not include small fish. The fishery product(s) may be in any form and may be raw (e.g., sashimi), filleted, SURIMI (e.g., chikuwa, kamaboko), frozen, dried (e.g., dried fish), freeze-dried, or the like. The intake of fishery products in the subject of administration is the average value for one week prior to the start of intake of the composition of the present invention. In a preferred embodiment, the intake of fishery products in the subject of administration may be determined by a survey method using the Food Intake Frequency Questionnaire (Takahashi et al., “EIYOUGAKU ZASHI” (Journal of Nutrition), Vol. 59, No. 5, p. 221-232 (2001)). The questionnaire contains questions equivalent to the following: “What kind, how much, and how many times a week do you eat fishery products and processed products thereof? Please fill in a separate field for small fish. The normal amount is 80 g. Here, 80 g is equivalent to one fillet, one medium fish, five slices of sashimi, one piece of dried fish, one medium chikuwa or kamaboko, 15 clams in their shells, three to four medium shrimps, and half a squid”. In the response column, respondents are asked to choose one of the following for breakfast, lunch, and dinner: none, a little (½ of the normal amount), normal, and plenty (1.5 times the normal amount), and to indicate the number of times per week they eat them.

In one embodiment, the subject of administration of the composition of the present invention may be a person whose fishery product intake is 0 kcal/day or more and 71 kcal/day or less, for example, 60 kcal/day or less, 50 kcal/day or less, 40 kcal/day or less, 30 kcal/day or less, 20 kcal/day or less, or 10 kcal/day or less. Alternatively, the subject of administration in the present invention may be a person whose fishery product intake is 0 kcal/kg body weight/day or more and 1.1 kcal/kg body weight/day or less, for example, 0.89 kcal/kg body weight/day or less, 0.75 kcal/kg body weight/day or less, 0.60 kcal/kg body weight/day or less, 0.45 kcal/kg body weight/day or less, 0.30 kcal/kg body weight/day or less, or 0.15 kcal/kg body weight/day or less.

The composition of the present invention may be prepared in a form suitable for pharmaceutical compositions, food compositions (e.g., functional foods, health foods, supplements, etc.), food additives, and others: for example, the composition may be prepared in a form of any of various solid formulations such as granules (including dry syrup), capsules (soft capsules, hard capsules), tablets (including chewables, etc.), powder (powdery preparations), rounds; liquid formulations such as oral liquids (including liquid, suspension, syrup); or the like. For example, the composition of the present invention can be formulated as soft capsules with the active ingredient filled in a gelatin coating.

Examples of additives for formulation include excipients, lubricants, binders, disintegrants, fluidizers, dispersants, wetting agents, preservatives, viscosity agents, pH adjusters, coloring agents, flavor and odor modifying agents, surfactants, dissolution aids, or the like. In the case of a liquid form, a thickener such as pectin, xanthan gum, guar gum, or the like may be blended. It can also be made into coated tablets using a coating agent or into a paste-like agglutinant. Furthermore, even in the case of preparation in other forms, conventional methods should be followed.

The composition of the present invention may have a form of a food composition or food additive. As used herein, the food composition means all foods including beverages, and includes general foods including health foods such as supplements, as well as foods for specified health uses and nutrition functional foods as defined in the Health Functional Foods System of the Consumer Affairs Agency. For example, a functional food with an indication of having an endurance-enhancing effect is provided. For example, fish oil-containing foods can be provided as they are. The food composition or food additive of the present invention also includes food materials for imparting an endurance-enhancing effect by adding it to, mixing it with, or applying it onto other foods. In addition to foods, it can also be provided as animal feeds, or the like.

The food composition or food additive of the present invention may be prepared by blending the composition of the present invention in a food composition or food additive, thereby imparting an endurance-enhancing function.

The food composition of the present invention may be provided as, for instance, beverages, confectioneries, breads, or soups. Examples include: general retorted foods, frozen foods, instant foods (e.g., noodles), canned foods, sausages, cookies, biscuits, cereal bars, crackers, snacks (e.g., potato chips), pastries, cakes, pies, candies, chewing gum (including pellets and sticks), jellies, soups, ice cream, dressings, yogurt, etc.; supplements in the form of, for instance, tablets, capsules, emulsions, etc.; soft drinks; or the like. The method of producing these foods is not limited as long as it does not impair the effects of the present invention, and should follow the procedure used by those skilled in the art for each food.

It is included in the scope of the present invention to market the product of the present invention by indicating the effect of the composition of the present invention on the packaging container, product instructions, or pamphlets. The scope of the present invention also includes advertising and selling the product of the present invention by displaying the effects of the present invention on television, Internet websites, pamphlets, newspapers, magazines, and other media. When the composition of the present invention is used as a pharmaceutical or quasi-drug, the efficacy and effect of endurance enhancement may be indicated on, for instance, the packaging container.

The amount of the active ingredient ingested by the subject in the present invention is not particularly limited, and the amount equal to or greater than the effective amount to obtain the desired effect, for example, is ingested. The effective amount to obtain the desired effect is herein the dose required to enhance endurance. For example, in the case of adults, the active ingredient can be ingested at a dose of 10 mg/kg body weight/day or more, e.g., 50 mg/kg body weight/day or more, or 100 mg/kg body weight/day or more, depending on conditions such as the subject's age, body weight, and health conditions. The active ingredient of the composition of the present invention does not have strong side effects. Therefore, the daily intake is not limited, but may be, for example, 500 mg/kg body weight/day or less, 400 mg/kg body weight/day or less, 200 mg/kg body weight/day or less, or 150 mg/kg body weight/day or less. Similarly, the period of intake of the composition of the present invention is not limited, but may be, for example, 4 weeks or more, 5 weeks or more, 6 weeks or more, 7 weeks or more, or 8 weeks or more. In addition, the intake may be continued until at least an improvement in endurance is observed.

In one embodiment, the composition of the present invention may be used such that a subject ingests, for 8 weeks or longer, a highly unsaturated fatty acid at 2.0 g/day or less, e.g., 1.9 g/day or less, 1.8 g/day or less, 1.7 g/day or less, 1.6 g/day or less, 1.5 g/day or less, 1.4 g/day or less, or 1.3 g/day or less. Alternatively, the composition of the present invention may be used such that a subject ingests, for 8 weeks or longer, a highly unsaturated fatty acid at 30 mg/kg body weight/day or less, e.g., 28 mg/kg body weight/day or less, 27 mg/kg body weight/day or less, 26 mg/kg body weight/day or less, 25 mg/kg body weight/day or less, 24 mg/kg body weight/day or less, 23 mg/kg body weight/day or less, 22 mg/kg body weight/day or less, 21 mg/kg body weight/day or less, 20 mg/kg body weight/day or less, or 19 mg/kg body weight/day or less. Alternatively, the composition of the present invention may be used such that a subject ingests, for 8 weeks or longer, a highly unsaturated fatty acid at 19 mg/kg body weight/day or more and 30 mg/kg body weight/day or less, e.g., 28 mg/kg body weight/day or less, 27 mg/kg body weight/day or less, 26 mg/kg body weight/day or less, 25 mg/kg body weight/day or less, 24 mg/kg body weight/day or less, 23 mg/kg body weight/day or less, 22 mg/kg body weight/day or less, 21 mg/kg body weight/day or less, or 20 mg/kg body weight/day or less.

In one embodiment, in the case where the highly unsaturated fatty acid is eicosapentaenoic acid and/or docosahexaenoic acid, the composition of the present invention may be used such that a subject ingests, for 8 weeks or longer, the highly unsaturated fatty acid at 1.0 g/day or less, e.g., 0.95 g/day or less, 0.90 g/day or less, or 0.86 g/day or less. Alternatively, in the case where the highly unsaturated fatty acid is eicosapentaenoic acid and/or docosahexaenoic acid, the composition of the present invention may be used such that a subject ingests, for 8 weeks or longer, the highly unsaturated fatty acid at 15 mg/kg body weight/day or less, e.g., 14 mg/kg body weight/day or less or 13 mg/kg body weight/day or less. Alternatively, in the case where the highly unsaturated fatty acid is eicosapentaenoic acid and/or docosahexaenoic acid, the composition of the present invention may be used such that a subject ingests, for 8 weeks or longer, the highly unsaturated fatty acid at 13 mg/kg body weight/day or more and 15 mg/kg body weight/day or less, e.g., 14 mg/kg body weight/day or less. Here, the amount of highly unsaturated fatty acid intake is the value obtained by converting the constituent fatty acids in the triglyceride, namely the active ingredient of the composition of the present invention, to free fatty acids. As shown in PTL 7, it has been known that the transfer of EPA into the blood was transiently enhanced upon a single intake of EPA/MCT structured lipid. However, it is not known whether or not there is a relationship between blood EPA levels and endurance enhancement.

In a preferred embodiment, the composition of the present invention is an endurance-enhancing composition comprising, as an active ingredient, a triglyceride containing a highly unsaturated fatty acid and a medium-chain fatty acid as constituent fatty acids, wherein
the highly unsaturated fatty acid accounts for 10 to 70 wt % in fatty acid makeup of the composition and the medium-chain fatty acid accounts for 20 to 70 wt % in fatty acid makeup of the composition;
the highly unsaturated fatty acid is eicosapentaenoic acid and docosahexaenoic acid;
the medium-chain fatty acid is caprylic acid and capric acid;
the composition is used such that 13 mg/kg body weight/day or more and 15 mg/kg body weight/day or less of the highly unsaturated fatty acid is ingested by a subject for 8 weeks or longer;
the subject is a person whose “high-intensity physical activity days at work per week” are from 0 to 2 days, and whose “medium-intensity physical activity days at work per week” are from 0 to 3 days, and whose “days with travel of 10 minutes or longer per week” are from 4 to 7 days, which are question items of the GPAQ; and
enhancement of the endurance is at least one selected from the group consisting of prolonged exercise duration, prolonged exercise duration during high-intensity exercise, prolonged exercise duration during aerobic exercise, prolonged exercise duration during incremental load exercise, accelerated lipid burning during exercise, accelerated lipid burning during high-intensity exercise, accelerated lipid burning during aerobic exercise, accelerated lipid burning during incremental load exercise, increased anaerobic exercise threshold, reduced fatigue during exercise, and enhanced muscle endurance.

In a preferred embodiment, the composition of the present invention is an exercise duration-prolonging composition comprising, as an active ingredient, a triglyceride containing a highly unsaturated fatty acid and a medium-chain fatty acid as constituent fatty acids, wherein
the highly unsaturated fatty acid accounts for 10 to 70 wt % in fatty acid makeup of the composition and the medium-chain fatty acid accounts for 20 to 70 wt % in fatty acid makeup of the composition;
the highly unsaturated fatty acid is eicosapentaenoic acid and docosahexaenoic acid;
the medium-chain fatty acid is caprylic acid and capric acid;
the composition is used such that 13 mg/kg body weight/day or more and 15 mg/kg body weight/day or less of the highly unsaturated fatty acid is ingested by a subject for 8 weeks or longer;
the subject is a person whose “high-intensity physical activity days at work per week” are from 0 to 2 days, and whose “medium-intensity physical activity days at work per week” are from 0 to 3 days, and whose “days with travel of 10 minutes or longer per week” are from 4 to 7 days, which are question items of the GPAQ; and
the fishery product intake of the subject is 0 kcal/kg body weight/day or more and 1.1 kcal/kg body weight/day or less, 0.89 kcal/kg body weight/day or less, 0.75 kcal/kg body weight/day or less, 0.60 kcal/kg body weight/day or less, 0.45 kcal/kg body weight/day or less, 0.30 kcal/kg body weight/day or less, or 0.15 kcal/kg body weight/day or less.

In one aspect, the present invention may provide a method of enhancing endurance, comprising causing a subject to ingest an effective amount of the composition of the present invention. The method may be performed in accordance with the description herein regarding ingestion or administration of the composition of the present invention.

### EXAMPLES

Hereinbelow, the present invention will be described in more detail with reference to Examples. However, the present invention is not limited to them.

### Example 1

### 1. Materials and Methods

### (1) Preparation of Test Substance

Refined fish oil (28 wt % EPA and 12 wt % DHA as standard values; manufactured by Nissui) and triglycerides exclusively containing medium-chain fatty acids as constituent fatty acids (MCT; about 60 wt % caprylic acid and about 40 wt % capric acid) were used. The refined fish oil and MCT were mixed at a weight ratio of 55:45, and sodium methoxide was added at 0.2 wt %. The mixture was then stirred at 60-80° C. for 60 minutes for transesterification. The product was washed with water, dried, decolorized, and deodorized to prepare triglycerides containing fish oil-derived fatty acids including EPA and medium-chain fatty acids as constituent fatty acids (hereinafter, referred to as STG in the Examples).

A mixed oil obtained by mixing the refined fish oil and MCT at a weight ratio of 50:50 (in the Examples below, PM) was prepared as a control sample.

Table 1 shows the fatty acid makeup of the refined fish oil, MCT, STG, and PM.

TABLE 1

| | MCT | Fish oil | PM | STG |
|---|---|---|---|---|
| | (wt %) | | | |
| C8:0 | 57.9 | 0.7 | 26.9 | 25.7 |
| C10:0 | 42.1 | 0.6 | 20.4 | 19.5 |
| C12:0 | 0.1 | 0.1 | --- | --- |
| C14:0 | --- | 5.3 | 2.7 | 2.8 |
| C15:0 | --- | 0.3 | 0.1 | 0.2 |
| C16:0 | --- | 6.4 | 3.3 | 3.6 |
| C16:1 | --- | 9.1 | 4.7 | 4.8 |
| C16:2 | --- | 1.8 | 0.9 | 1 |
| C16:3 | --- | --- | --- | --- |
| C16:4 | --- | 4.5 | 2.6 | 2.7 |
| C17:0 | --- | 0.1 | --- | --- |
| C17:1 | --- | 0.1 | --- | --- |
| C18:0 | --- | 0.6 | 0.3 | 0.3 |
| C18:1 | --- | 8.2 | 4.8 | 4.9 |
| C18:2n-6 | --- | 1.2 | 0.6 | 0.6 |
| C18:3n-3 | --- | 0.3 | 0.6 | 0.7 |
| C18:4n-3 | --- | 4.0 | 2.3 | 2.4 |
| C20:1 | --- | 0.5 | 0.3 | 0.3 |
| C20:2n-6 | --- | 0.1 | --- | --- |
| C20:4n-6 | --- | 1.5 | 0.5 | 0.5 |
| C20:4n-3 | --- | 1.0 | 0.6 | 0.6 |
| C20:5n-3 | --- | 28.5 | 15.6 | 16.1 |
| C21:5n-3 | --- | 1.1 | 0.6 | 0.6 |
| C22:5n-3 | --- | 2.6 | 1.6 | 1.6 |
| C22:5n-6 | --- | 0.4 | 0.2 | 0.2 |
| C22:6n-3 | --- | 12.8 | 7.1 | 7.3 |
| C24:1 | --- | 0.1 | --- | --- |
| Others | --- | 8.1 | 3.3 | 3.6 |
| Total | 100 | 100 | 100 | 100 |

### (2) Study Participants

Nineteen healthy young men without regular training habits were enrolled in this study.

Data (mean±SD) on subjects are presented below. The age, body weight, mean BMI, and VO₂peak (maximum oxygen uptake) are data at the beginning of the study. The number of days of physical activity was the mean value of one week before the start of the study and was based on the answers to the Global Physical Activity Questionnaire (GPAQ; Bull F C et al., Global physical activity questionnaire (GPAQ): nine country reliability and validity study. J Phys Act Health. 2009 November; 6 (6): 790-804). Here, the “work” includes schoolwork and part-time jobs. In addition, the terms “high-intensity physical activity” and “moderate-intensity physical activity” refer to physical activity of 8 and 4 METs, respectively (Global Physical Activity Questionnaire (GPAQ) Analysis Guide, Surveillance and Population-Based Prevention, Prevention of Noncommunicable Diseases Department, World Health Organization). The intake of fishery products was a value averaged over one week prior to the start of the study and was determined by a survey method using the Food Intake Frequency Questionnaire (Takahashi et al., “EIYOUGAKU ZASHI” (Journal of Nutrition), Vol. 59, No. 5, p. 221-232 (2001)). The questionnaire had the following questions: “What kind, how much, and how many times a week do you eat fishery products and processed products thereof? Please fill in a separate field for small fish. The normal amount is 80 g. Here, 80 g is equivalent to one fillet, one medium fish, five slices of sashimi, one piece of dried fish, one medium chikuwa or kamaboko, 15 clams in their shells, three to four medium shrimps, and half a squid”. In the response column, respondents were asked to choose one of the following for breakfast, lunch, and dinner: none, a little (½ of the normal amount), normal, and plenty (1.5 times the normal amount), and to fill in the number of times per week they eat them.
Age: 20±2 years old
Body weight: 67.1±8.9 kg
Mean BMI: 22.8±3.1 kg/m²
VO₂peak (maximum oxygen uptake)*: 43.4±6.1 mL/kg/min
“High-intensity physical activity days at work per week”: 0.4±0.9 days
“Medium-intensity physical activity days at work per week”: 1.1±1.7 days
“Days with travel of 10 minutes or longer per week”: 5.3±1.0 days
*: VO₂peak measured in the Examples can be treated as almost the same value as VO₂max (maximum oxygen uptake).

### (3) Study Summary

Study participants were randomly divided into two groups: one receiving STG (STG group; n=9) and the other receiving PM (PM group; n=10). The STG and PM groups received 4.38 g/day of STG and PM, respectively, for 8 weeks. Both of the groups would ingest 600 mg/day of EPA and 260 mg/day of DHA in terms of free fatty acids.

The study participants were subjected to a maximum oxygen uptake (VO₂peak) test before and 8 weeks after intake of the test substance. During the study period, the study participants were instructed not to change their diet habits prior to the intake. The average calorie intake of the study participants during the study period was 2235 kcal/day; the average water intake was 1085 g/day; the average protein intake was 90 g/day; the average lipid intake was 74 g/day; and the average carbohydrate intake was 289 g/day.

### (4) Maximum Oxygen Uptake (VO₂Peak) Measuring Method

A multistep load incremental method using a bicycle ergometer (pedal rotation speed during exercise was set at 60 rpm) was used for measurement. After 3 minutes of rest, 3 minutes of warm-up exercise at 20-watt load was performed. After the warm-up exercise, the exercise was started at a 60-watt load, and the load was increased by 30 watts every 2 minutes until fatigue/exhaustion. The final exercise load was defined as the load when the pedal rotation speed of the study participant fell below 50 rpm three times, and the exercise and measurement were ended at the time the measurement of oxygen uptake at that load was completed. The exercise duration was recorded at the end of the exercise.

In addition, the carbon dioxide output was also measured in this study. It has been known that oxygen uptake increases in proportion to exercise load, but carbon dioxide output increases rapidly after a certain exercise load. Thus, the point at which the carbon dioxide output increased rapidly was defined as the anaerobic exercise threshold (ventilatory exercise threshold). The change in anaerobic exercise threshold was evaluated by calculating (oxygen uptake at anaerobic exercise threshold/maximum oxygen uptake)×100 (% VO₂peak; AT level).

### (5) Statistical Analysis

The exercise duration and AT levels were analyzed by two-way analysis of variance. The change in exercise duration, change in AT level, and change in VO₂peak were analyzed by a corresponding t-test.

### [Results]

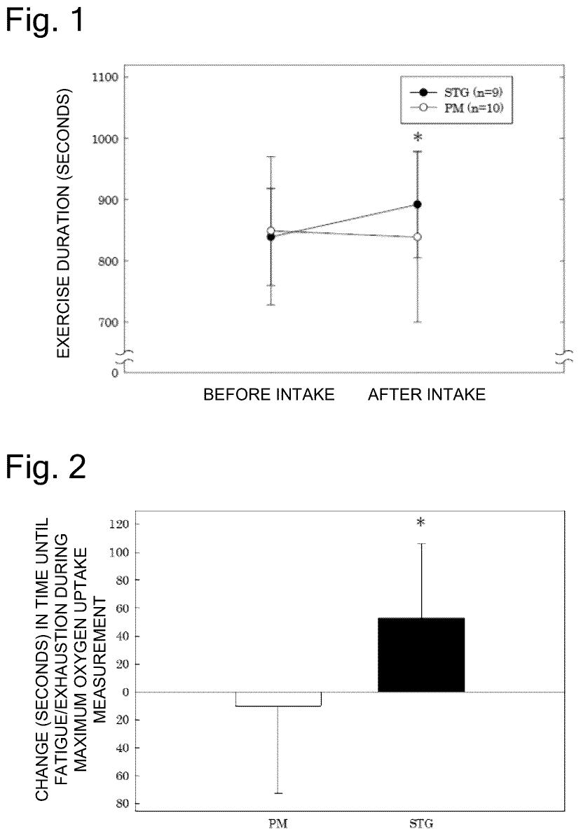

FIG. 1 shows the exercise duration during maximum oxygen uptake measurement before and after intake in each group. In the STG group, the exercise duration after STG intake was significantly increased when compared to that before STG intake (two-way analysis of variance, p<0.05). On the other hand, in the PM group, there was no significant change in exercise duration between before and after PM intake. The change (53±53 seconds) in exercise duration between before and after intake in the STG group and the change (−10±63 seconds) in exercise duration between before and after intake in the PM group were analyzed by t-test. As a result, a significant difference (p<0.05) was observed (FIG. 2). Therefore, the triglyceride containing a highly unsaturated fatty acid and a medium-chain fatty acid as constituent fatty acids has been demonstrated to have the function of enhancing endurance, especially prolonging the exercise duration.

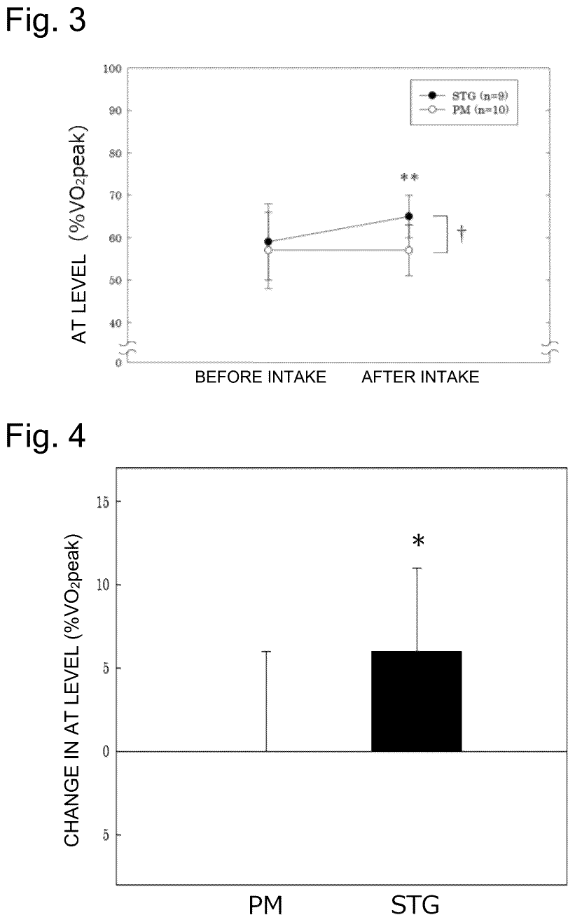

FIG. 3 shows values for anaerobic exercise threshold (% VO₂peak) before and after intake in each group. In the STG group, the AT level was significantly higher after STG intake when compared to before STG intake and compared to after PM intake (two-way analysis of variance, p<0.05). On the other hand, in the PM group, there was no significant change in AT level between before and after PM intake. The change (6±5) in AT level between before and after intake in the STG group and the change (0±6) in AT level between before and after intake in the PM group were analyzed by t-test. As a result, a significant difference (p<0.05) was observed (FIG. 4). Higher AT levels mean prolonged aerobic exercise allowable duration. Carbohydrates and lipids are the main energy sources during aerobic exercise. Carbohydrates are the main energy source during anaerobic exercise. AT levels are expected to be higher due to increased lipid utilization during exercise. Therefore, the results in the Examples have suggested that intake of triglycerides containing a highly unsaturated fatty acid and a medium-chain fatty acid as constituent fatty acids promotes the utilization of lipids as an energy source during exercise more than the case of intake of physically mixed oil with similar fatty acid makeup.

In addition, a stratified analysis of the exercise duration in the STG group was also performed by dividing the STG group into two groups: those with lower (n=5) and those with higher (n=3) fishery product intake than the mean of the study participants in the STG group (71 kcal/day, or 1.1 kcal/kg body weight/day) (Table 2). As a result, it has been found that the group with low fishery product intake tended to exhibit a greater change in the exercise duration between before and after STG intake than the group with high fishery product intake.

TABLE 2

| | Exercise duration | | Exercise duration | | | |
|---|---|---|---|---|---|---|
| | (seconds) before | | (seconds) after | | Change in exercise | |
| | intake | | intake | | duration (seconds) | |
| | | Standard | | Standard | | Standard |
| | Mean | deviation | Mean | deviation | Mean | deviation |
| Subjects (n = 5) with lower | 812.2 | 77.7 | 897.8 | 77.5 | 85.6* | 39.8 |
| fishery product intake than | | | | | | |
| mean of the entire subjects | | | | | | |
| Subjects (n = 3) with higher | 910.0 | 6.6 | 938.7 | 22.7 | 28.7 | 26.0 |
| fishery product intake than | | | | | | |
| mean of the entire subjects | | | | | | |

*p = 0.072

Therefore, the triglyceride containing a highly unsaturated fatty acid and a medium-chain fatty acid as constituent fatty acids has been demonstrated to elicit a markedly higher endurance-enhancing function on subjects with lower fishery product intake.

### INDUSTRIAL APPLICABILITY

The present invention provides a pharmaceutical composition, a food composition, a food additive, etc., that enhances endurance.

1.-11. (canceled)

12. A method of enhancing endurance, comprising feeding a subject with an effective amount of a composition including, as an active ingredient, a triglyceride containing a highly unsaturated fatty acid and a medium-chain fatty acid as constituent fatty acids.

13. The method according to claim 12, wherein the highly unsaturated fatty acid accounts for 10 to 70 wt % in fatty acid makeup of the composition and the medium-chain fatty acid accounts for 20 to 70 wt % in fatty acid makeup of the composition.

14. The method according to claim 12, wherein 19 mg/kg body weight/day or more and 30 mg/kg body weight/day or less of the highly unsaturated fatty acid is ingested by a subject for 8 weeks or longer.

15. The method according to claim 12, wherein the subject has a fishery product intake of 1.1 kcal/kg body weight/day or less.

16. The method according to claim 12, wherein the subject has a maximum oxygen uptake of 60 mL/kg/min or less.

17. The method according to claim 12, wherein the highly unsaturated fatty acid is a fatty acid with 20 or more carbon atoms.

18. The method according to claim 17, wherein the highly unsaturated fatty acid is eicosapentaenoic acid or docosahexaenoic acid.

19. The method according to claim 12, wherein the medium-chain fatty acid is a C₈₋₁₂ fatty acid.

20. The method according to claim 12, wherein enhancement of the endurance is at least one selected from the group consisting of prolonged exercise duration, prolonged exercise duration during high-intensity exercise, prolonged exercise duration during aerobic exercise, prolonged exercise duration during incremental load exercise, accelerated lipid burning during exercise, accelerated lipid burning during high-intensity exercise, accelerated lipid burning during aerobic exercise, accelerated lipid burning during incremental load exercise, increased anaerobic exercise threshold, reduced fatigue during exercise, and enhanced muscle endurance.

21. The method according to claim 12, wherein the composition is provided in a form of a food composition or food additive.
